(19) United States
(12) Patent Application Publication
(10) Pub. No.: US 2022/0258143 A1
(43) Pub. Date: Aug. 18, 2022

(54) HYBRID MATERIAL COMPRISING A PROTEIN MATRIX AND COPPER NANOPARTICLES THEREIN, PROCESS FOR PREPARING THE SAME AND USE THEREOF

(71) Applicants: CONSEJO SUPERIOR DE INVESTIGACIONES CIENTÍFICAS (CSIC), Madrid (ES); SAMSUNG ELECTRONICS CO., LTD., Gyeonggi-do (KR)

(72) Inventors: Jose Miguel PALOMO CARMONA, Madrid (ES); Noelia LOSADA GARCÍA, Madrid (ES); Alba RODRÍGUEZ OTERO, Madrid (ES)

(21) Appl. No.: 17/612,411

(22) PCT Filed: May 25, 2020
(86) PCT No.: PCT/EP2020/064407

§ 371 Date: Nov. 18, 2021

(30) Foreign Application Priority Data
May 24, 2019 (ES) P201930459

(51) Int. Cl. B01J 31/00; C12N 9/20; B01J 27/055; B01J 35/00; B01J 37/04; B01J 37/06; B01J 37/36; B01J 37/16

(52) U.S. Cl. CPC B01J 31/003; C12N 9/20; C12Y 301/01003; B01J 27/055; B01J 37/16; B01J 37/04; B01J 37/06; B01J 37/36; B01J 35/006

(57) ABSTRACT

The present invention relates a hybrid material comprising: a protein matrix comprising the lipase B from Candida antarctica and nanoparticles of copper species selected from: Cu (0), Cu₂O Cu₃(PO₄)₂ or any combination thereof, having their larger dimension between 3 and 15 nm. The material was prepared by the use of an enzyme in a buffer solution and a copper salt at room temperature. The material shows excellent catalase-like activity, excellent catalytic capacity in the degradation of organic pollutants, like Bisphenol A, and in the reduction of 4-nitrophenol to 4-aminophenol.

The invention relates to a hybrid material that comprises a protein matrix and copper nanoparticles therein. The present invention also describes the synthesis thereof by a single and green technology. The hybrid material can be used as a catalyst, since it shows excellent catalase-like activity, excellent catalytic performance in the degradation of the organic pollutant Bisphenol A and in the reduction of 4-nitrophenol (pNP) to 4-aminophenol (pAP).

### STATE OF ART

Copper is an earth-abundant and low-cost metal, which has been described very useful for different applications, such as antimicrobial agent, environmental remediation [1], electronics and specially in catalysis. Indeed, it has been a very extensive work in the recent years in the area of application of Cu catalysts in chemical reactions, especially in C—C bond (click chemistry, C—H activation, etc.), selective oxidation, reductions.

In particular, Cu nanoparticles have generated a great deal of interest in recent years. [2] The high surface-to-volume ratio of nanomaterials compared to bulk materials generally makes them attractive candidates for their application as catalysts.

Actually, it has been described different practical and straightforward multiple ways of preparing Cu nanomaterials.[3]

However, the use of Cu nanoparticles (NPs) is restricted by Cu's inherent instability under atmospheric conditions, which makes it prone to oxidation. Many efforts to develop the methods and supporting materials that increase the stability of Cu NPs by altering their sensitivity to oxygen, water, and other chemical entities has encouraged the exploration of alternative Cu-based NPs with more complex structures, such as core/shell Cu NPs or systems based on copper oxides.

The synthesis of Cu and copper oxide NPs essentially centers around mainly four chemical reaction types, reduction, oxidation, hydrolysis or condensation. Depending on the choice of final materials, either one or a combination of aforementioned chemistries can be applied. The synthesis of Cu NPs often entails the reduction of Cu(I) or Cu(II) sources. The synthesis of copper oxide NPs, on the other hand, basically requires hydrolysis of the precursors followed by a dehydration process leading to the final materials. Additionally, an oxidation process (sometimes unavoidable for Cu-based NPs) can be deployed for the preparation of Cu based NPs with higher oxidation numbers from their respective precursors of lower oxidation states. In synthetic processes, the techniques that are applied provide a suitable environment and energy to facilitate the process of choice while additional constraints are imposed to modulate the stability, properties, and morphology of the final NPs.

Therefore, the development of methodologies to preparing highly active, selective, stable and robust copper nanoparticles is mandatory. [4] Also sustainable systems, inexpensive and high amount CuNPs is also desirable from a more applicable point of view.

The copper species in term of catalysis is one of the important aspects on the final properties of the nanocatalysts in a particular reaction. Therefore, the development of technology which permit to control the synthesis specifically of one copper specie represents an important challenge.

One important application where Cu catalysts has been utilized in the last years has been focused on the degradation of organic pollutants, and specifically Bisphenol A (BPA).

BPA is an important monomer in the manufacture of polycarbonate plastics, food cans, and other daily used chemicals. Daily and worldwide usage of BPA and BPA-contained products led to its ubiquitous distribution in water, sediment/soil, and atmosphere. Moreover, BPA has been identified as an environmental endocrine disruptor for its estrogenic and genotoxic activity. [5] Thus, BPA contamination in the environment is an increasingly worldwide concern, and methods to efficiently remove BPA from the environment are urgently recommended. In 2018, the European Commission adopted a proposal to strengthen the regulation on the use of BPA in food contact materials, the new Regulation introduces a new specific migration limit (SML) of 0.05 ppm of BPA.

Therefore, a methodology to obtain a very fast and efficient and green strategy to eliminate BPA is mandatory. Actually only a few examples using Cu or Cu-based materials has been applied on this reaction in many cases using homogeneous catalysts. [6]

In this invention excellent results in completed BPA degradation in water has been achieved with the heterogeneous Cu bionanohybrids, which were reused several times without losing the activity, a critical role for industrial application.

The degradation of hydrogen peroxide (H₂O₂) in water and oxygen have an important reaction in biological systems. Indeed, the formation of H₂O₂ due to mitochondrial superoxide leakage perpetuates oxidative stress in neuronal injury. The biological catalyst involved in the elimination is Catalase, a copper metalloenzyme.

The application of this enzyme could be antioxidant therapy target however this is restricted by its labile nature and inadequate delivery.

Therefore, the development of stable and well-delivery artificial systems mimics this biological activity represents a challenge.

### REFERENCES

[1] Singh, J., Dutta, T., Kim, K.-H., Rawat, M., Samddar, P., Kumar, P. ‘Green’ synthesis of metals and their oxide nanoparticles: Applications for environmental remediation. J. Nanobiotechnol., 2018, 16, art. no. 84.
[2] Gawande, A. Goswami, M. B., Felpin F.-X., Asefa, T., Huang, X., Silva, R., Zou, X., Zboril, R., Varma, R. S. Cu and Cu-Based Nanoparticles: Synthesis and Applications in Catalysis. Chem. Rev. 2016, 116, 3722-3811.
[3] Zaera, F. Nanostructured Materials for Applications in Heterogeneous Catalysis. Chem. Soc. Rev. 2013, 42, 2746-2762.
[4] Ben Aissa, M. A., Tremblay, B., Andrieux-Ledier, A., Maisonhaute, E., Raouafi, N., Courty, A. Copper Nanoparticles of Well-Controlled Size and Shape: A New Advance in Synthesis and Self-Organization. Nanoscale 2015, 7, 3189-3195.
[5] Ulutao, O. K., Yildiz N, Durmaz, E, Ahbab M. A., Barlas N, Åok Ã. An in vivo assessment of the genotoxic potential of bisphenol A and 4-tert-octylphenol in rats. Arch Toxicol 2011, 85, 995-1001.
[6] Pachamuthu, M. P., Karthikeyan, S., Maheswari, R., Lee, A. F., Ramanathan, A. Fenton-like degradation of Bisphenol A catalyzed by mesoporous Cu/TUD-1. Appl. Surf. Sci., 2017, 393, 67-73.

### DESCRIPTION OF THE INVENTION

The present invention discloses a hybrid material comprising a protein matrix and nanoparticles (NPs) of Cu species and also discloses the process for preparing thereof. The material shows excellent catalase-like activity, excellent catalytic capacity in the degradation of organic pollutants, like Bisphenol A (BPA) and in the reduction of 4-nitrophenol (pNP) to 4-aminophenol (pAP).

Then, a first aspect of the present invention relates to a hybrid material comprising:
a protein matrix comprising the lipase B from Candida antarctica and
nanoparticles of copper species selected from: Cu (0), Cu₂O, Cu₃(PO₄)₂ or any combination thereof,
   wherein the nanoparticles have an average diameter between 3 and 15 nm and are homogeneously distributed within the matrix.

The lipase B from Candida Antarctica (CALB) (GenBank reference number: CAA83122.1) is a non-specific lipase from Candida antarctica. CALB is stable over a broad pH range, especially in the alkaline pH range. This enzyme exhibits a high degree of substrate specificity, allowing large groups on the carboxylic acid and resulting in highly regio- and enantioselective conversions.

The lipase B from Candida Antarctica is commercially available from, for example, by Novozymes under the name of Lipozyme® CALB.

The interactions between the matrix and the nanoparticles are non-covalent.

The matrix is formed by the lipase B from Candida antarctica in the form of aggregate. The term “aggregate” is a multiplicity of protein molecules that have become grouped through steric interaction or otherwise with one another.

In a preferred embodiment of the invention, the matrix consists of lipase B from Candida antarctica.

In a preferred embodiment, the hybrid material has between 22 and 94% by weight in Cu, obtained by elemental analysis ICP-OES (inductively coupled plasma optical emission spectrometry).

A second aspect of the present invention relates to a process for preparing the hybrid material described in the first aspect of the present invention. The process comprises the next steps:

a) addition under stirring of the lipase B from Candida antarctica to a buffer solution, wherein the pH of the buffer solution ranges between 7 and 10
b) addition of a copper salt to the solution obtained in step a) at room temperature (20-25° C.)
c) incubation of the solution obtained in step b) for a time between 16 h and 3 days,
d) collecting, washing and drying the hybrid material obtained in the previous step.

In a preferred embodiment, the collected hybrid material (that is formed as a solid in the solution) is washed with water and/or drying by lyophilization in step d).

In a preferred embodiment, the process includes an additional step c)′ (after step c) and before step d)) which can be:
a reduction step comprising the addition of a reducing agent, preferably sodium borohydride, to the mixture obtained in step c) or
an oxidation step comprising the addition of an oxidant agent, preferably hydrogen peroxide or NaOH, to the mixture obtained in step c) or

In a preferred embodiment, the reduction or the oxidation step is carried out for about 30 min.

Preferably, the buffer solution (that is an aqueous solution) of step a) is either a sodium phosphate buffer or a sodium bicarbonate buffer; more preferably, a phosphate buffer 0.1 M (pH 7) or a sodium bicarbonate buffer 0.1 M (pH 10).

In a preferred embodiment, between 0.3 to 3 mg of lipase B from Candida antarctica is added per ml of buffer solution, more preferably 18 mg in 60 mL of buffer solution in step a).

Preferably, the copper salt is Cu₂SO₄, more preferable Cu₂SO₄.5H₂O.

In a preferred embodiment, 10 mg of copper salt is added in step b) per ml of buffer solution.

Preferably, the incubation time in step c) ranges from 16 h to 24 h, more preferably 16 h. Incubation means that the mixture is left under stirring for the specified time.

In a preferred embodiment, in step c′) the reducing agent, preferably sodium borohydride, is added to a final concentration of at least 0.012 M in the mixture, more preferably, between 0.012 and 0.12 M, even more preferably to a final concentration of 0.12.

In an embodiment, the process for preparing the hybrid material comprises:

a) addition under stirring of the lipase B from Candida antarctica to a buffer solution of sodium bicarbonate 0.1 M (pH 10), wherein the protein is added in a proportion of 0.3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for 16 h.
c′) addition of sodium borohydride to a final concentration thereof of 0.12 M,
d) collecting, washing with water and drying by lyophilization the hybrid material obtained on the previous step.

This embodiment provides a hybrid material having Cu (0) as the main copper species and containing around 20% by weight of Cu(I) in form of Cu₂O. The nanoparticles are of around 9 nm size (in the present invention, the term “size” refers to average diameter). The content of Cu in the material is around 84% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 0.3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for 16 h.
c′) addition of sodium borohydride to a final concentration thereof of 0.12 M,
d) collecting, washing with water and drying by lyophilization the hybrid material obtained on the previous step.

In this case the main Cu species was Cu (I) in form of Cu₂O around 70% by weight and also containing around 30% by weight of Cu (0). This hybrid material present 81% by weight Cu and the nanoparticles are of 15 nm.

In another embodiment, the process for preparing the hybrid material comprises:

a) addition under stirring of the lipase B from Candida antarctica to a buffer solution of sodium bicarbonate 0.1 M (pH 10), wherein the protein is added in a proportion of 0.6 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for 16 h.
c′) addition of sodium borohydride to a final concentration thereof of 0.12 M,
d) collecting, washing with water and drying by lyophilization the hybrid material obtained on the previous step.

In this case, a hybrid material having Cu (0) as the main copper species is also obtained. The nanoparticles are of around 6 nm size. The content of Cu in the material is around 94% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 0.6 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for 16 h.
c′) addition of sodium borohydride to a final concentration thereof of 0.12 M,
d) collecting, washing with water and drying by lyophilization the hybrid material obtained on the previous step.

In this embodiment, differences in Cu species and nanoparticles size were observed. In this case, hybrid material comprises exclusively Cu₂O species, without traces of Cu (0). The nanoparticles are crystalline and of around 10 nm size. The content of Cu in the material is around 61% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 0.3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for a 16 h.
d) collecting, washing with water and drying by lyophilization the hybrid material obtained on the previous step.

In this case the copper species obtained is Cu₃(PO₄)₂. The nanoparticles are of around 3-5 nm size. The content of Cu in the material is around 32% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 0.3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for a 16 h.
c′) addition of H₂O₂ to a final concentration thereof of 0.1 M,
d) collecting, washing with water and drying by lyophilization the hybrid material obtained on the previous step.

In this case the copper species obtained is Cu₃(PO₄)₂. The nanoparticles are of around 5 nm size. The content of Cu in the material is around 22% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 0.3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for a 16 h. c′) addition of NaOH to a final concentration thereof of 0.5 M,
d) collecting, washing with water and drying by lyophilization the hybrid material obtained on the previous step.

In this case the copper species obtained is Cu₃(PO₄)₂. The nanoparticles are of around 6 nm size. The content of Cu in the material is around 35% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 0.3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for a 16 h.
c′) addition of sodium borohydride to a final concentration thereof of 0.012 M,
d) collecting, washing with water and drying by lyophilization the hybrid material obtained on the previous step.

At these synthetic conditions, the hybrid material comprises extremely crystalline Cu₃(PO₄)₂ nanoparticles (size approx. 10 nm). Also, the Cu content in this case is around 48% by weight.

In another embodiment, the process for preparing the hybrid material comprises:

a) addition under stirring of the lipase B from Candida antarctica to a buffer solution of sodium phosphate 0.1 M (pH 7), wherein the protein is added in a proportion of 3 mg per ml of buffer solution,
b) addition of a copper salt, preferably Cu₂SO₄.5H₂O, to the solution obtained in step at room temperature (20-25° C.), wherein the copper salt is added in a proportion of 10 mg per ml of buffer solution,
c) incubation of the solution obtained in step b) for 3 days.
c′) optionally addition of sodium borohydride to a final concentration thereof of 0.12 M,
d) collecting, washing with water and drying by lyophilization the hybrid material obtained on the previous step.

In these cases, Cu₃(PO₄)₂ is the main copper species, containing also Cu₂O specially where reduction step was carried out. A content of Cu of around 50% by weight is obtained.

The present invention also relates to the hybrid material obtained by the process described in the second aspect of the invention. The hybrid material obtained by the process of the invention has the features of the hybrid material described in the first aspect of the present invention.

Another aspect of the present invention refers to the use of the hybrid material described in the present invention as a catalyst.

In a preferred embodiment, the hybrid material described in the present invention is used as a catalyst for degrading organic pollutants, preferably phenolic compounds; more preferably for degrading Bisphenol-A, forming CO₂ and H₂O.

In another preferred embodiment, the hybrid material described in the present invention is used for the reduction of 4-nitrophenol (pNP) to 4-aminophenol (pAP).

In another preferred embodiment, the hybrid material described in the present invention is used for the degradation of H₂O₂, forming H₂O and O₂, since the hybrid material has catalase-like activity.

In the present invention, the terms “hybrid material”, “biohybrid material”, “bionanohybrid material” or simply “biohybrid” or bionanohybrid” or “hybrid material comprising a protein matrix and copper nanoparticles therein” are used as synonyms and refer to a material comprising a matrix of lipase B from Candida antarctica, (organic compound) and Cu species (inorganic compound) selected from: Cu (0), Cu₂O Cu₃(PO₄)₂ or any combination thereof in the form of nanoparticles having an average diameter between 3 and 15 nm.

The term “average” of a dimension of a plurality of particles means the average of that dimension for the plurality. For example, the term “average diameter” of a plurality of nanoparticles means the average of the diameters of the nanoparticles, where the diameter of a single nanoparticle is the average of the diameters of that nanoparticle. The nanoparticles are spherical or substantially spherical. The average diameter is obtained by transmission electron microscopy (TEM).

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples and drawings are provided by way of illustration and are not intended to be limiting of the present invention.

### BRIEF DESCRIPTION OF THE DRAWINGS

FIG. 1: Characterization of the Cu-CALB-BIC biohybrid prepared in the example 1 of the present invention. A) XRD spectrum, B) TEM images.

FIG. 2: Characterization of the Cu-CALB-PHOS biohybrid prepared in the example 1 of the present invention. A) XRD spectrum, B) TEM images.

FIG. 3: Characterization of the Cu-CALB-BIC2 biohybrid prepared in the example 1 of the present invention. A) XRD spectrum, B) TEM images.

FIG. 4: Characterization of the Cu-CALB-PHOS2 biohybrid prepared in the example 1 of the present invention. A) XRD spectrum, B) TEM images.

FIG. 5: Characterization of the Cu-CALB-PHOS-NR biohybrid prepared in the example 1 of the present invention. A) XRD spectrum, B) TEM images.

FIG. 6: Characterization of the Cu-CALB-PHOS-NRNaOH biohybrid prepared in the example 1 of the present invention. A) XRD spectrum, B) TEM images. C) C) HTEM images.

FIG. 7: Characterization of the Cu-CALB-PHOS-NRH₂O₂ biohybrid prepared in the example 1 of the present invention. A) XRD spectrum, B) TEM images. C) HTEM images.

FIG. 8: Characterization of the Cu-CALB-PHOS10% biohybrid. A) XRD spectrum, B) TEM images.

FIG. 9: Characterization of the Cu-CALB-PHOS-10 biohybrid. A) XRD spectrum, B) SEM image.

FIG. 10: Characterization of the Cu-CALB-PHOS-NR-10 biohybrid. A) XRD spectrum, B) SEM image.

FIG. 11: Representation of degraded percentage of BPA catalyzed by different Cu-CALB nanohybrids at 100 mM phosphate buffer pH 8 in the presence of 100 mM of H₂O₂.

FIG. 12: Representation of degraded percentage of BPA catalyzed by different hybrids in the presence of 100 mM of H₂O₂ at different pHs. A) Cu-CALB-PHOS2 and B) Cu-CALB-PHOS.

FIG. 13: Degradation of BPA by Cu-CALB-PHOS2 at 100 mM phosphate buffer pH 8 in the presence of different concentrations of H₂O₂.

FIG. 14: Recycling of Cu-CALB-PHOS2 in oxidative degradation of BPA at 100 mM phosphate buffer pH 8 in the presence of 100 mM of H₂O₂.

FIG. 15: A. Representation of degraded percentage of BPA (4.6 ppm) catalyzed by different Cu-CALB-PHOS2 at 100 mM phosphate buffer pH 8 in the presence of 100 mM of H₂O₂. B. Proposed mechanism of BPA degradation.

FIG. 16: Catalase activity of Cu-CALB-PHOS2. A) degradation of 50 mM of H₂O₂ in distilled water adjusted at different pHs. B) degradation of 50 mM of H₂O₂ at different phosphate buffer concentration at pH 6.

FIG. 17: Scheme of preparation of the bionanohybrids comprising Cu species NPs and a protein matrix.

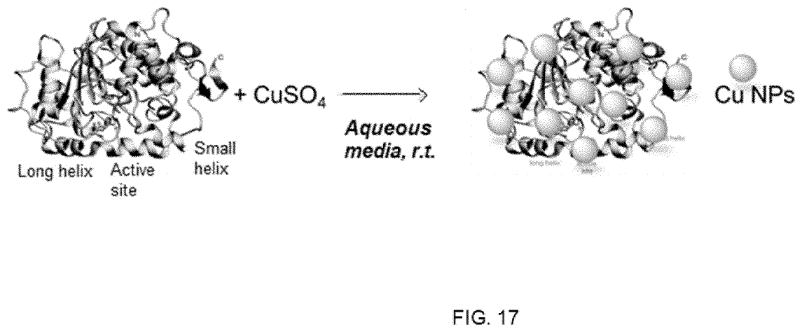

### EXAMPLES

Some examples carried out by the inventors are provided below in order to illustrate the present invention:

### Experimental Section

General

Lipase B from Candida antarctica (CAL-B) solution (Lipozyme®CALB) (10 mg/mL) was purchased from Novozymes (Copenhagen, Denmark). Copper (II) sulfate pentahydrate [Cu₂SO₄×5H₂O] and hydrogen peroxide (33%) were from Panreac (Barcelona, Spain). P-nitrophenol, p-nitrophenyl propionate, sodium bicarbonate, sodium phosphate, sodium borohydride and Bisphenol A (BPA) were purchased from Sigma-Aldrich (St. Louis, Mo., USA). HPLC grade acetonitrile was purchased from Scharlab (Barcelona, Spain). Inductively coupled plasma-optical emission spectrometry) (ICP-OES) was performed on a OPTIMA 2100 DV instrument (PerkinElmer, Waltham, Mass., USA). X-Ray diffraction (XRD) patterns were obtained using a Texture Analysis D8 Advance Diffractometer (Bruker, Billerica, Mass., USA) with Cu Kα radiation. Transmission electron microscopy (TEM) and high resolution TEM microscopy (HRTEM) images were obtained on a 2100F microscope (JEOL, Tokyo, Japan) equipped with an EDX detector INCA x-sight (Oxford Instruments, Abingdon, UK). Interplanar spacing in the nanostructures was calculated by using the inversed Fourier transform with the GATAN digital micrograph program (Corporate Headquarters, Pleasanton, Calif., USA). Scanning electron microscopy (SEM) imaging was performed on a TM-1000 microscope (Hitachi, Tokyo, Japan). To recover the biohybrids, a Biocen 22 R (Orto-Alresa, Ajalvir, Spain) refrigerated centrifuge was used. Spectrophotometric analyses were run on a V-730 spectrophotometer (JASCO, Tokyo, Japan). A spectrum P100 HPLC system (Thermo Scientifics, Waltham, Mass., USA) was used. Analyses were run at 25° C. using an L-7300 column oven (Hitachi, Tokyo, Japan) and a UV6000LP detector (Thermo Scientifics, Waltham, Mass., USA).

### Example 1: General Synthesis of Cu-CALB-BIC and Cu-CALB-PHOS Bionanohybrids

1.8 mL (18 mg protein) of commercial Candida antarctica lipase solution was added to 60 mL buffer 0.1M (sodium bicarbonate pH=10 or sodium phosphate pH 7) in a 250 mL glass bottle containing a small magnetic bar stirrer. Then, 600 mg of Cu₂SO₄×5H₂O (10 mg/ml) was added to the protein solution and it was maintained for 16 hours. After the first 30 min incubation, the solution turned cloudy (turquoise) and the pH solution was measured indicating a decrease from 8 or 6 depending on the buffer used. After 16 h, 6 mL of NaBH₄ (300 mg) aqueous solution (1.2 M) was added to the cloudy solution (in two times of 3 mL) obtaining a final concentration of 0.12 M of sodium borohydride in the mixture. The solution turned rapidly black and, the mixture was reduced during 30 min. After the incubation, in all cases, the mixture was centrifuged at 8000 rpm for 5 min, (10 mL per falcon type tube). The generated pellet was re-suspended in 15 mL of water. The pH of the supernatant solution was measured to be approximately 7 or 9. It was centrifuged again at 8000 r.p.m for 5 min and the supernatant removed. The pH of the supernatant solution was measured again, given a pH value of 7. The process was repeated twice more. Finally, the supernatant was removed and the pellet of each falcon was re-suspended in 2 mL of water, collected all solutions in a round-bottom flask, frozen with liquid nitrogen and lyophilized for 16 hours. After that 150 mg of the so called Cu-CALB-BIC and Cu-CALB-PHOS respectively were obtained.

Different modifications of the protocol were made to obtain different species. Initially, a catalyst was prepared using double amount of enzyme (3.6 mL CALB solution instead of 1.8 mL), obtaining Cu-CALB-PHOS-2 and Cu-CALB-BIC-2.

Another variation of the protocol was used, in which the reduction step was not performed, obtaining Cu-CALB-PHOS-NR. In addition to the last variation, an oxidation step was also performed instead of reduction step by the addition of either 6 ml of a 500 mM solution of sodium hydroxide (NaOH) for 30 min or 6 ml of a 0.1 M solution of hydrogen peroxide (H₂O₂) for 30 min (60 μL of the H₂O₂ stock solution in 6 mL of distillate water), obtaining Cu-CALB-PHOS-NRNaOH and Cu-CALB-PHOS-NRH₂O₂ respectively. Another one of the variations was reduction of sodium borohydride at 10%, adding 6 mL of water containing NaBH₄ (30 mg), obtaining Cu-CAL-B-PHOS10% R.

The last variation was referred both to the enzyme amount as well as the incubation time, increasing them from 1.8 mL to 18 mL in enzyme volume and from 16 h to 72 h; the obtained catalysts were Cu-CALB-PHOS-10 and Cu-CALB-PHOS-NR-10.

Characterization of the different Cu bionanohybrids was performed by XRD, ICP-OES, TEM and SEM analysis.

### Example 2: Catalytic Reduction of 4-Nitrophenol (pNP) to 4-Aminophenol (pAP)

To an aqueous solution of p-nitrophenol (pNP) (1 mM; 2 mL), solid NaBH₄ (3 mg) was added to reach a final concentration of 0.04 M (The typical catalytic reaction was performed by adding an excess of NaBH₄ (0.04 M) to ensure its constant concentration throughout the reaction and, therefore, to apply a pseudo-first-order kinetic with respect to the pNP to an aqueous solution of the substrate in the presence of catalysts). In these conditions, upon the addiction of NaBH₄, the initial absorbance band of the solution of pNP undergoes to an immediate shift from 317 to 400 nm due to the formation of 4-nitrophenolate ions. Immediately after that, 3 mg of the different Cu-CALB bionanohybrids were added under gentle stirring at 25° C. in an orbital shaker. The reaction progress was monitored by taking out an aliquot of the solution (0.1 mL) at different times, diluting it with distilled water (2 mL) and measuring the absorption spectrum between 500 and 300 nm in a PMMA cuvette (Table 1).

TABLE 1
Degradation of pNP by all bionanohybrids.

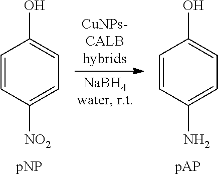 [SMILES: O=[N+]([O-])c1ccc(O)cc1 ; Nc1ccc(O)cc1]

| | pNP | |
|---|---|---|
| | Degradation | Time |
| Bionanohybrid | Yield (%) | (min) |
| Cu-CALB-PHOS | 100 | 1.5 |
| Cu-CALB-BIC | 100 | 0.5 |
| Cu-CALB-PHOS-2 | 100 | 3 |
| Cu-CALB-BIC-2 | 100 | 0.5 |
| Cu-CALB-PHOS-NR | 100 | 4 |
| Cu-CALB-PHOS-NR-NaOH | 100 | 3 |
| Cu-CALB-PHOS-NR-H₂O₂ | 100 | 3 |
| Cu-CALB-PHOS10% R | 100 | 1.5 |
| Cu-CALB-PHOS-10 | 100 | 12.5 |
| Cu-CALB-PHOS-NR-10 | 100 | 15 |

### Example 3: Catalytic Degradation of Bisphenol-A (BPA)

A solution of 10 mM of BPA in pure acetonitrile was prepared. 0.2 ml of this solution were dissolved in 10 mL of either 100 mM or 5 mM sodium phosphate buffer pH 6, pH 7 or pH 8 to achieve a 0.2 mM concentration of BPA. The solution pH was adjusted using HCl or NaOH 1 M. Hydrogen peroxide was added to this BPA solution to obtain different concentrations (12, 25, 50, 100 or 150 mM). To initialize the reaction, 3 mg of the nanohybrid was added to 2 mL of this solution (BPA and H₂O₂) in a 7 mL glass flask. Gentle stirring was provided at room temperature by a roller. Samples (30 μl) at different times were taken and the reaction was followed by HPLC. The samples were diluted 5 times in a mixture of distilled water/acetonitrile 50/50 before injection. The HPLC column was C8 Kromasil 150×4.6 mm AV-2059. The HPLC conditions used were: an isocratic mixture of 50% acetonitrile and 50% bi-distilled water, UV detection at 225 nm using a Diode array detector, and a flow rate of 1 mL/min. Under these conditions, the retention time of BPA was 4.90 min, and for H₂O₂ was 1.57 min.

### Example 4: Reuse of Cu-CALB-PHOS2 Nanohybrid in the Degradation of Bisphenol-A (BPA)

The Cu-CALB-PHOS2 nanohybrid was reused in five cycles for the degradation of BPA using the conditions described above. The catalyst was washed with water several times and centrifuged before the next reaction.

### Example 5: Catalase-Like Activity of Cu Nanohybrids

A substrate solution was prepared adding 52 μL of hydrogen peroxide to 9.8 mL of 100 mM or 5 mM phosphate buffer (pH 6, pH 7 and pH 8) or distilled water in order to obtaining a final concentration of 50 mM. The solution pH was adjusted using HCl or NaOH 1 M. To start the reaction, 4.5 mg of the Cu nanohybrid or 50 μL of Catazyme® 25 L (1 mg/mL in distilled water) was added to a 3 mL of the previous solution at room temperature. The reaction was followed by measuring the degradation of hydrogen peroxide recording the decrease of absorbance by spectrophotometrically at 240 nm in quartz cuvettes of 1 cm path length, adding 2 mL of this solution at different times. After each measurement the volume added was again recovered and poured to the reaction solution.

In order to determine the catalase activity for each catalyst, the ΔAbs/min value was calculated using the linear portion of the curve (ΔAbsₛ).

The specific activity (U/mg) was calculated using the following equation:

$$U ( μmol / min ) = \frac{Δ A b s}{t ( min )} \cdot \frac{{V}_{f} ( mL )}{ε ( \frac{mL}{μmol \cdot cm} ) \cdot b ( cm )}$$

where the molar extinction coefficient (ε) used was 43.6 M⁻¹cm⁻¹

### Results and Discussion:

1) Preparation and Characterization of Different Bionanohybrids Comprising the Protein Lipase B from Candida antarctica (CAL-B) and NPs of Different Cu Species:

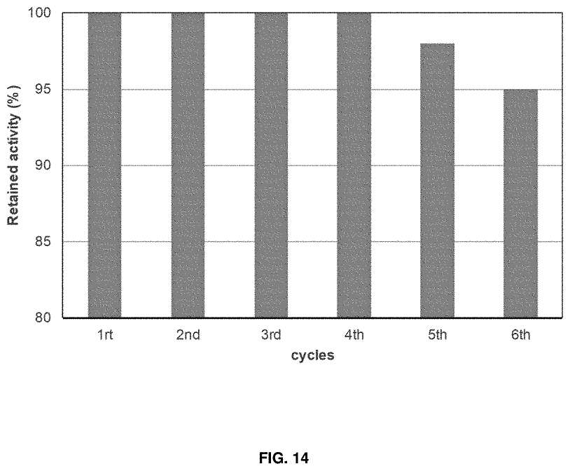

The synthesis of these bionanohybrids containing copper nanoparticles have been performed in aqueous media by adding the commercial lipase B from Candida antarctica (CAL-B, 33 kDa, monomeric enzyme, supplied by Novozymes) to an aqueous solution of fully water-soluble copper salt at room temperature and under gentle stirring (FIG. 14). The copper (II) sulfate pentahydrate [Cu₂SO₄×5H₂O] was used as copper salt with a concentration of 10 mg/mL. 1.8 mL of CAL-B was added to 60 mL of copper salt solution at room temperature. Different buffers and different concentrations were tested and the use of 100 mM of sodium bicarbonate (pH 10) and 100 mM of sodium phosphate (pH 7) were the best options.

After 16 h incubation, the solid was reduced by using sodium borohydride, washed several times with distilled water, centrifuged and lyophilized overnight to obtaining the two heterogeneous biohybrids Cu-CALB-BIC and Cu-CALB-PHOS respectively. Both catalysts were characterized by different analysis techniques such as XRD, ICP-OES, TEM and SEM. Different species were obtained depending on the buffer used in the synthesis.

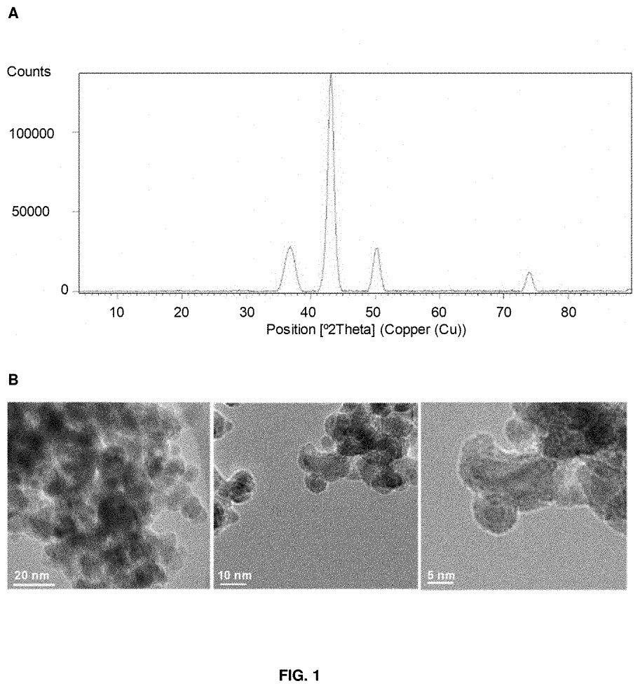

XRD analysis demonstrated that the main copper species in Cu-CALB-BIC hybrid was Cu(0), containing around 20% by weight of Cu(I) in form of Cu₂O (FIG. 1). TEM analysis showed the formation of nanoparticles of around 9 nm size (FIG. 1B). ICP-OES showed than the content of Cu in biohybrid Cu-CALB-BIC was 84% (The percentages of Cu in the hybrid material that are indicated in the present invention refers to percentages by weight). Using heating at 100° C. for drying instead of lyophilization, the Cu₂O increased to be around 60% of Cu species, the rest was Cu (0) (data not shown).

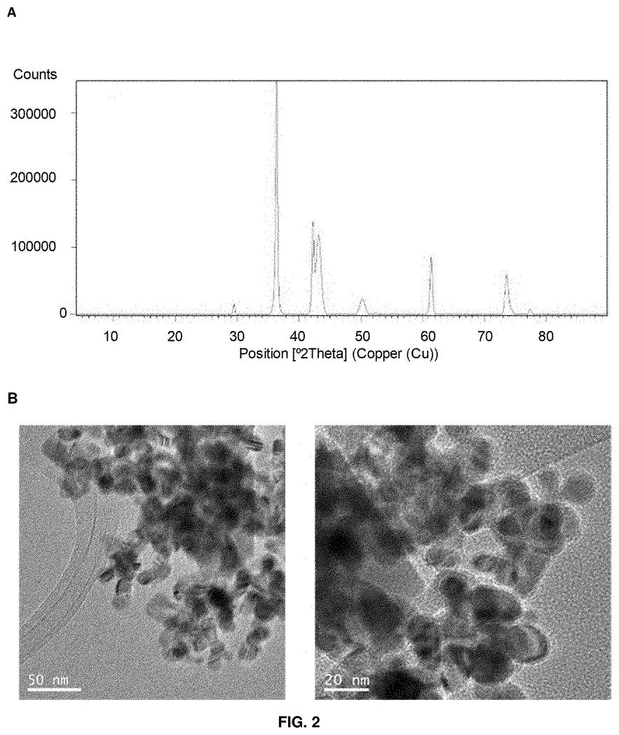

In the case of Cu-CALB-PHOS hybrid, XRD showed than the main Cu species was Cu(I) in form of Cu₂O around 70% and also containing around 30% of Cu (0) (FIG. 2). This biohybrid presented 81% Cu calculated by ICP analysis. TEM experiments revealed the formation of nanoparticles of 15 nm (FIG. 2B), slightly larger than with using bicarbonate as buffer of synthesis (FIG. 1). Also, this method was modified by using acetone in the washing step instead of water, or heating at 80° C. instead of lyophilization for obtaining the solid. In all these conditions the nanohybrid presented mixture of the different Cu species and the catalytic properties and mechanical stability of the solid were worse.

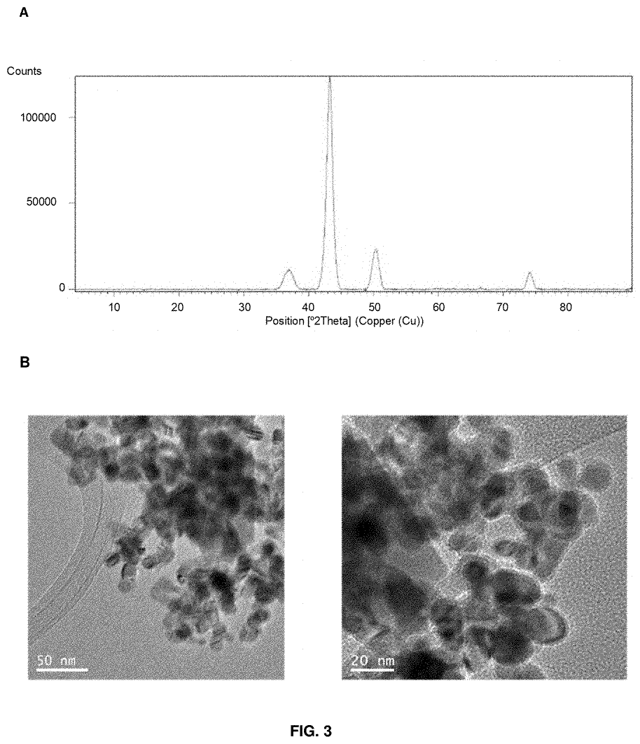

One modification on the previous protocols was adding the double amount of enzyme in the preparation maintaining the rest steps the same. This modification did not affect to the copper species in method using carbonate as buffer, where XRD pattern of the so-called nanohybrid Cu-CALB-BIC2 showed the characteristic peaks of Cu (0) and one minority at 37° of the Cu₂O (thus was slightly lower than in Cu-CALB-BIC) (FIG. 3). However, the content of Cu in this sample was 94%, around 10% more than using half amount of protein (Cu-CALB-BIC). TEM analysis revealed the formation of Cu (0)NPs as a core of 6 nm (FIG. 3B), smaller size than observed in Cu-CALB-BIC.

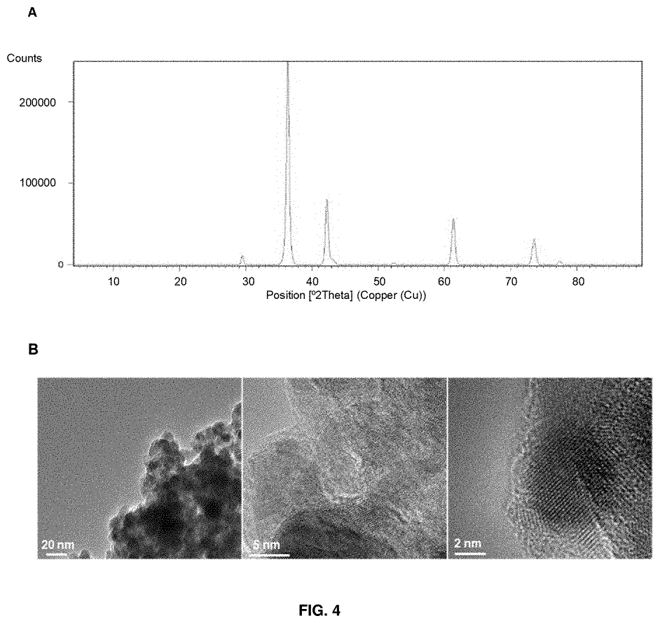

In the case of using phosphate as buffer, differences in Cu species and nanoparticles size were observed. In this case, XRD pattern determined that Cu-CALB-PHOS2 biohybrid showed peaks exclusively corresponding to Cu₂O species, without traces of Cu (0) (FIG. 4). Also changes in the amount of Cu was observed, being for this hybrid 61% determined by ICP-OES, 20% less than in Cu-CALB-PHOS. TEM analysis also exhibited the formation of crystalline nanoparticles with a diameter size of 10 nm (FIG. 4).

Therefore, by using this last methodology, a novel Cu bionanohybrid of controlled morphology, size and metal species was synthesized. This could be explained by the concept of larger amount of protein influence controlling the reduction of the cooper oxide species and also influenced in the coalescence step, controlling the nanoparticle growth.

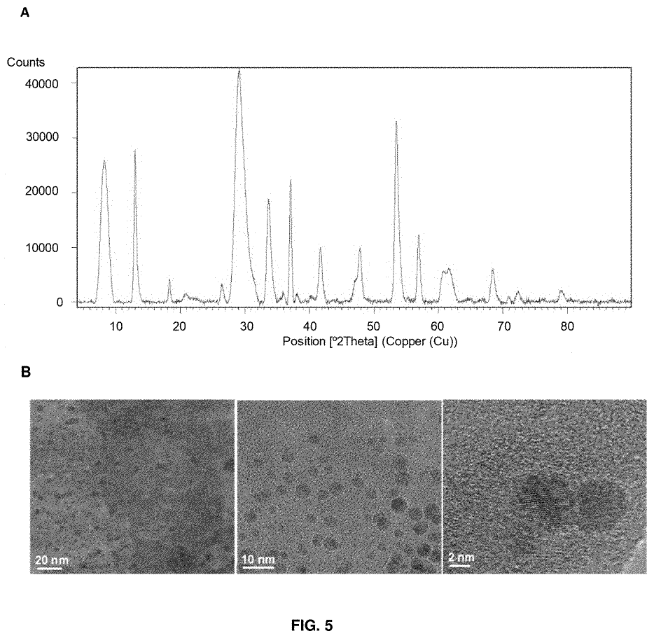

Furthermore, selecting the methodology using phosphate buffer, other types of Cu bionanohybrids were synthesized avoiding the reducing step (Cu-CALB-NR) or changing it by incubation in the presence of hydrogen peroxide (oxidative step) (Cu-CALB-NRH₂O₂) or NaOH (Cu-CALB-NRNaOH) in the methodology. In the three cases, a light blue solid was obtained instead of the typical black color for the other biohybrids. XRD showed that in this case the copper species were Cu₃(PO₄)₂ in all cases (FIGS. 5-7) and the Cu content were 32%, 22% and 35%, respectively.

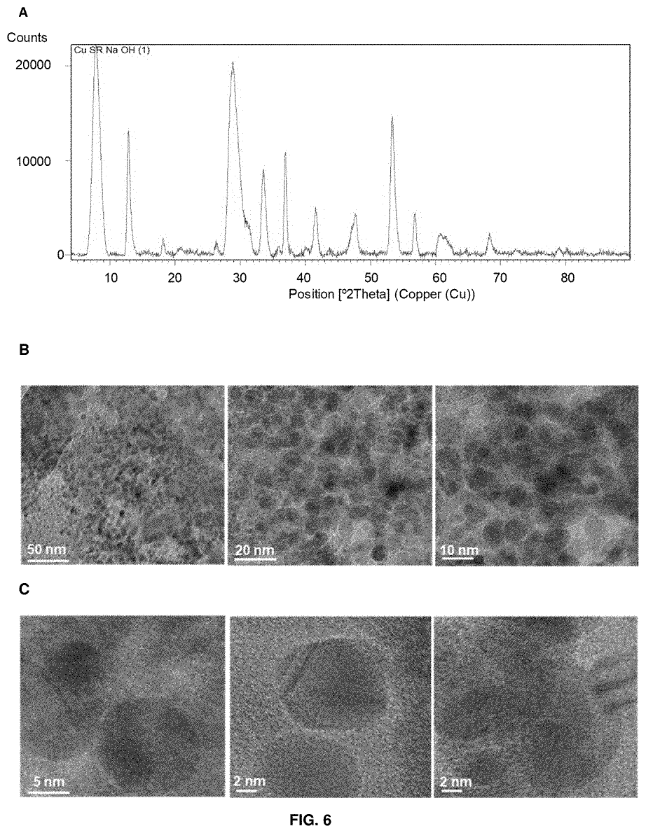

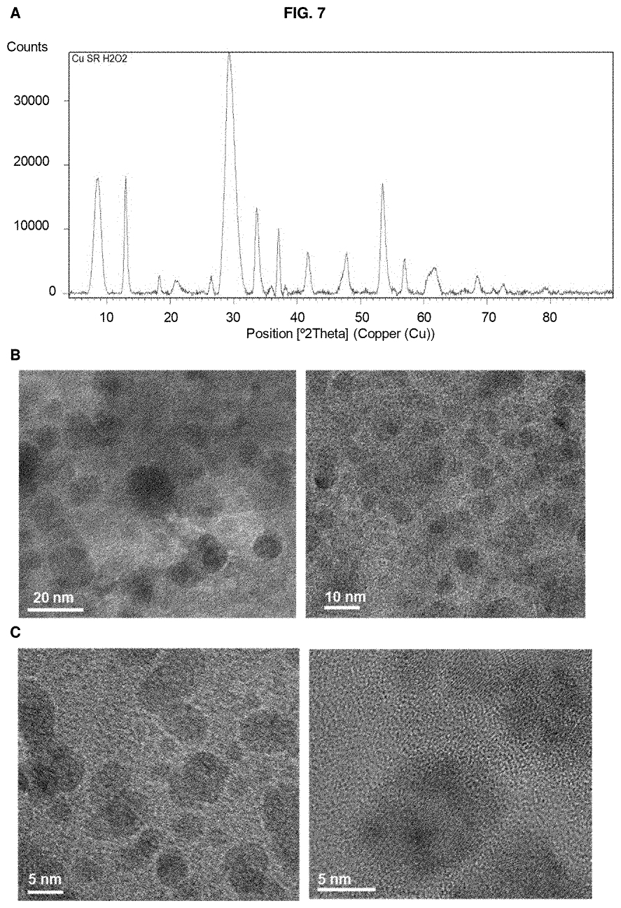

TEM analysis revealed the slightly differences on the diameter size of the nanoparticles. In all cases crystalline spherical nanoparticles were formed, from 3-5 nm in Cu-CALB-NR (FIG. 5), 5.8 nm in Cu-CALB-NRNaOH (FIG. 6) and 5 nm for Cu-CALB-NRH₂O₂ (FIG. 7).

Finally, a decrease in the amount of sodium borohydride used in the reduction step was performed. 10% (w/v) of NaBH₄ used in the previous method was added in this case maintaining the intact the rest of synthetic steps in the method of synthesis of Cu-CALB-PHOS, in this case the biohybrid called Cu-CALB-PHOS10% R.

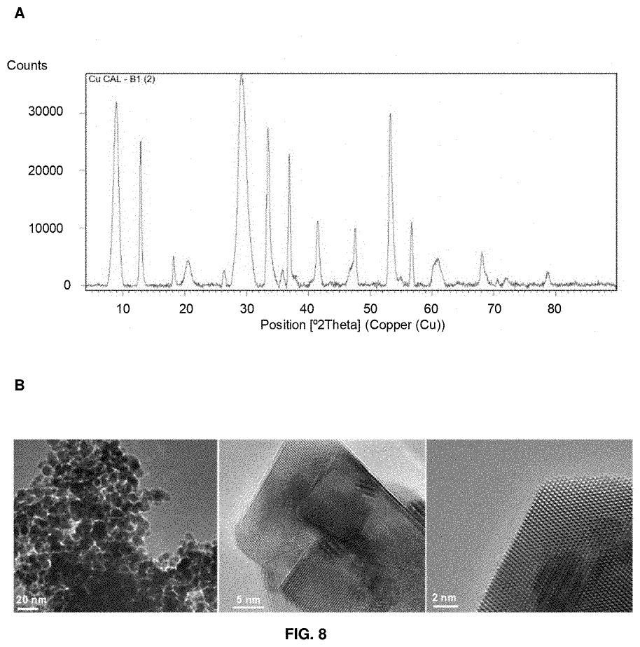

At these synthetic conditions, a biohybrid constitutes of extremely crystalline Cu₃(PO₄)₂ nanoparticles (diameter size approx. 10 nm) was obtained (FIG. 8) instead of the Cu/Cu₂O NPs biohybrid synthesized in Cu-CALB-PHOS (FIG. 2). Also, the Cu content in this case was 48% instead of 81% with the full reduction.

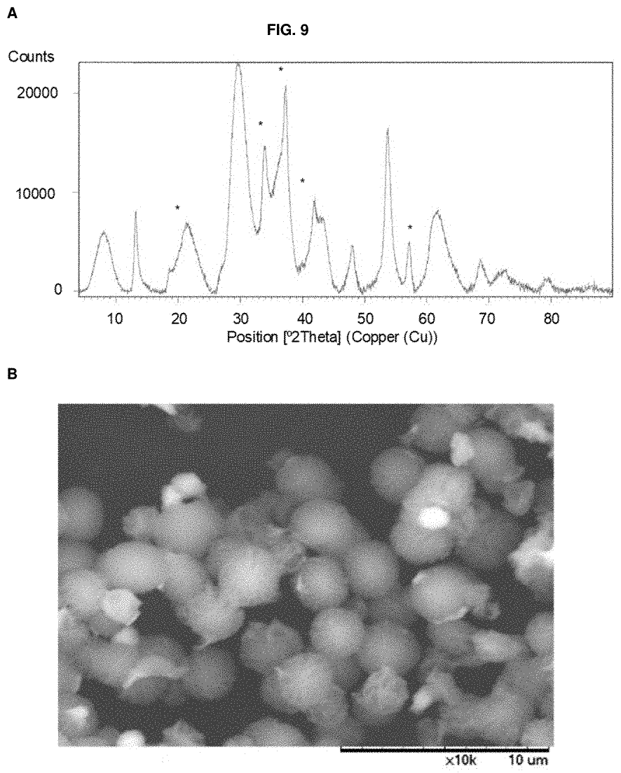

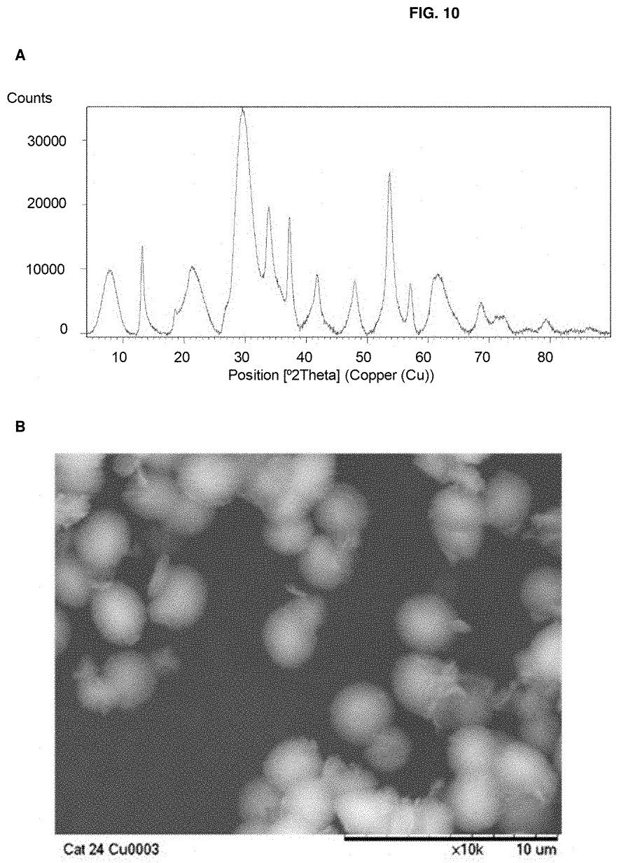

A final test using 10 times more amount of protein and increasing the incubation time from 1 day to 3 days was performed. These variations were used in two protocols, using phosphate buffer and with or without reduction step, being the Cu-CALB-PHOS-10 and Cu-CALB-PHOS-NR-10 bionanohybrids respectively. In this case SEM analysis revealed the formation of well-formed nanoflowers in both cases (FIG. 9-10). XRD pattern showed the presence of Cu₃(PO₄)₂ as main copper species, containing also Cu₂O specially in Cu-CALB-PHOS-10 where reduction step was used (FIG. 9). ICP-OES determined a content of cu of around 50% in each case.

In all cases, the bionanohybrids were synthesized by a very effective, simple and sustainable way at multimilligram scale, easily scalable to grams.

### 2) Determination of Catalytic Activity pNP Reduction

In order to evaluate the metallic activity of the novel bionanohybrids prepared in example 1, the activity of them in the reduction of p-nitrophenol (pNP) to p-aminophenol (pAP) in aqueous media and room temperature (r.t.) was performed (Table 1). The differences in the rate of degradation of pNP that exist between the different catalysts was due to the Cu species obtained.

Cu-CALB-BIC and Cu-CALB-BIC2 (mainly Cu (0)) were the faster nanocatalysts with a complete transformation of pNP to pAP (150 ppm) in 30 seconds. However, Cu nanohybrids containing Cu(I) species showed lower catalytic efficiency, Cu-CALB-PHOS2 was more active than Cu-CALB-PHOS which needs double of time to complete transformation, 3 min instead 1.5 min (Table 1).

The catalyst prepared without reduction steps or with very high amount of proteins showed lower catalytic activities, being necessary between 3 to 15 min for full degradation (Table 1).

### 3) Bisphenol A Degradation by Bionanohybrids

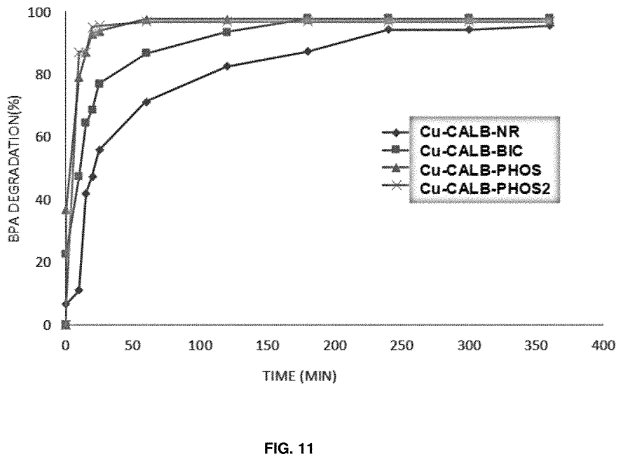

The catalytic capacity of the bionanohybrids prepared in example 1 was evaluated in the degradation of Bisphenol A (BPA) in aqueous media at room temperature. The experimental conditions selected were 100 mM buffer phosphate in range pH 6-8 and the amount of BPA was 46 ppm using H₂O₂ as oxidant. All the Cu nanocatalysts were initially tested using 100 mM of H₂O₂ in 100 mM phosphate buffer at pH 8. From them the results obtained for the best four ones are represented in FIG. 11. The best results were obtained using Cu-CALB-PHOS and Cu-CALB-PHOS2 where >95% BPA was degraded in 20 min. This represents, as far as we known, the fastest degradation process for eliminating BPA in aqueous media. The Cu-CALB-BIC hybrid degraded 68% BPA whereas Cu-CALB-NR only eliminated 47% at 20 min achieving >90% BPA degraded after 4 h incubation (FIG. 11). These results demonstrated that Cu₂O NPs species showed better catalytic activity than Cu (0) ones.

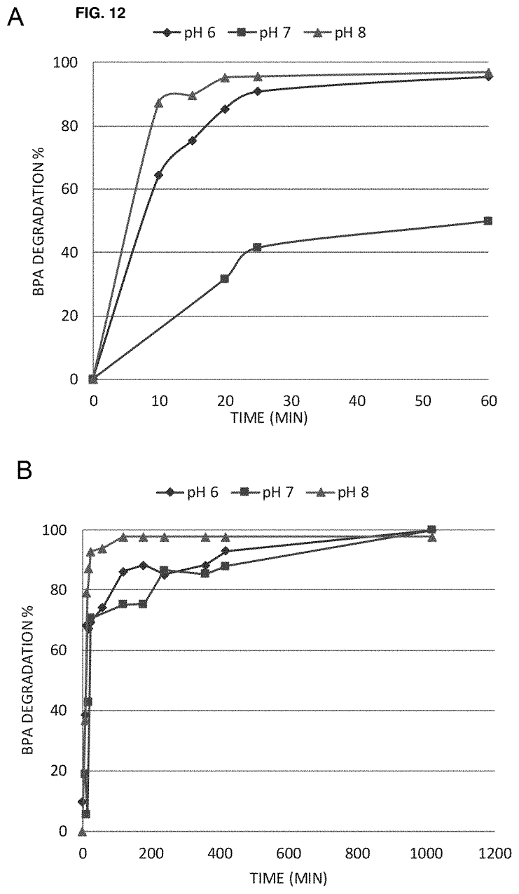

The reaction was also tested with these two excellent catalysts at different pHs (from 6 to 8) (FIG. 12). In both cases, Cu nanohybrids were better catalysts at pH 8, although surprisingly the efficiency for Cu-CALB-PHOS2 decreased at pH 7 more than pH 6 in comparison with Cu-CALB-PHOS where results were quite similar at these two pHs.

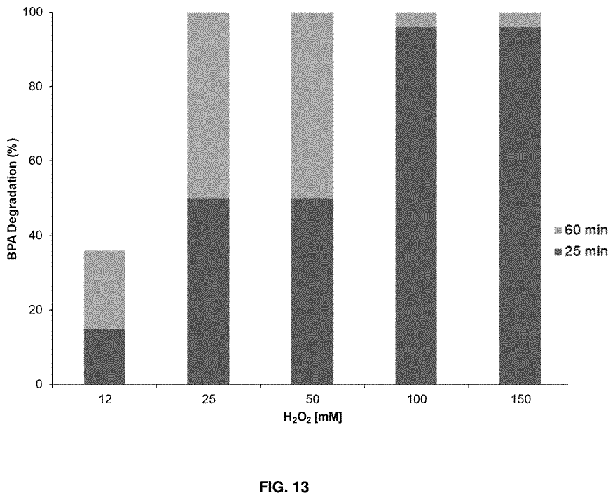

The amount of H₂O₂ also was evaluated. The reaction was performed using Cu-CALB-PHOS2 as catalyst at 100 mM phosphate pH 8 in the presence of different concentrations of H₂O₂ from 12 to 150 mM (FIG. 13). The results demonstrated that 100 mM seems to be the optimal amount for performing the reaction, because the use of higher amount did not accelerate the reaction and however the addition of less amount of H₂O₂ slowed down the reaction.

Once optimal conditions were obtained, a recycling experiment was performed using the best Cu bionanohybrid, Cu-CALB-PHOS2 (FIG. 14). The catalysts exhibited an excellent stability maintaining 95% of the catalytic efficiency after six cycles of use.

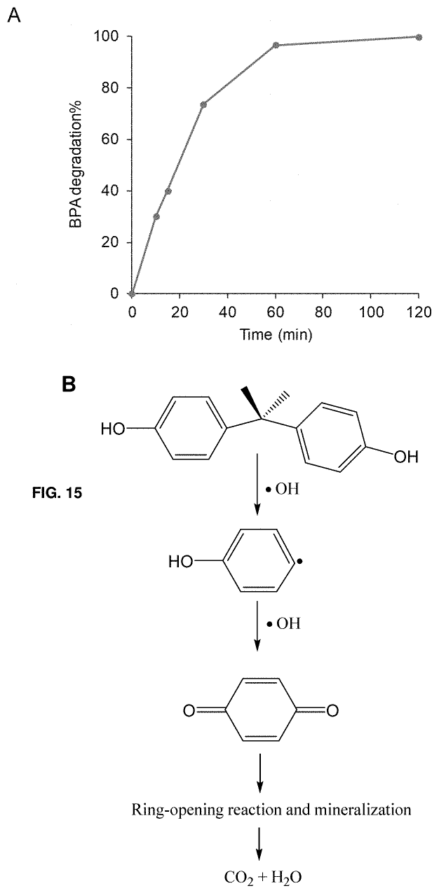

Considering the very fast degradation process by this catalyst and in order to elucidate a possible mechanism of degradation of BPA, the reaction was performed reducing the ratio mg catalyst/mL reaction volume from 1.5 (the previous result) to 0.3. At these conditions the catalyst still worked well, degrading more than 95% BPA in 60 min (FIG. 15A). Evaluating the intermediates points, it was possible to observed the formation of a mainly peak in HPLC corresponding to benzoquinone. Thus, a possible mechanism of the process can be by the formation of hydroxyl radical which is rapidly transformed mainly in benzoquinone which by ring-opening reaction follows a common degradation pathway to other intermediates peaks to finally degrade in CO₂ and H₂O (FIG. 15B), mechanism also described by Giu et al. (Gui, L., Jin, H., Zheng, Y., Peng, R., Luo, Y., Yu, P. Electrochemical Degradation of Bisphenol A Using Different Modified Anodes Based on Titanium in Aqueous Solution. Int. J. Electrochem. 2018, 13, 7141-7156).

### 4) Catalase-Like Activity of Cu-Nanoparticles Biohybrids

The catalase activity (degradation of H₂O₂) of the different synthesized Cu bionanohybrids in water, 5 mM buffer and 100 mM buffer at different pHs was evaluated (Table 2). Interesting differences were found between the different Cu bionanohybrids and specially in comparison with the native catalase from Novozymes (Catazyme).

TABLE 2
Catalase activities of the different Cu bionanohybrids (catalysts).

| | | Specific activity (U/mg) | | | |
|---|---|---|---|---|---|
| | | pH not | | | |
| Catalyst | Reaction solution | adjusted | pH 6 | pH 7 | pH 8 |
| Cu-CAL- | Distilled water | 0.43 | 0.92 | 0.37 | 0.37 |
| B-PHOS | 5 mM phosphate | | 0.23 | 0.43 | 1.29 |
| | buffer | | | | |
| | 100 mM phosphate | | 0.32 | 0.14 | 0.57 |
| | buffer | | | | |
| Cu-CAL- | Distilled water | 2.49 | 2.11 | 0.65 | 1.46 |
| B-PHOS2 | 5 mM phosphate | | 0.08 | 0.46 | 0.19 |
| | buffer | | | | |
| | 100 mM phosphate | | 0.38 | 0.35 | 0.08 |
| | buffer | | | | |
| Cu-CAL- | Distilled water | 1.19 | 0.36 | 1.02 | 0.20 |
| B-BIC | 5 mM phosphate | | 0.08 | 0.50 | 0.28 |
| | buffer | | | | |
| | 100 mM phosphate | | 0.17 | 0.19 | 0.06 |
| | buffer | | | | |
| Cu-CAL- | Distilled water | 0.65 | 0.65 | 0.79 | 0.79 |
| B-BIC2 | 5 mM phosphate | | 0.10 | 0.57 | 0.17 |
| | buffer | | | | |
| | 100 mM phosphate | | 0.02 | 0.17 | 0.10 |
| | buffer | | | | |
| Catazyme | Distilled water | 2.79 | 11.16 | 6.98 | 46.05 |
| | 5 mM phosphate | | 58.60 | 103.26 | 78.14 |
| | buffer | | | | |
| | 100 mM phosphate | | 26.51 | 36.28 | 9.77 |
| | buffer | | | | |

The Cu-CALB-PHOS2 biohybrid exhibited the highest catalytic activity in the degradation of hydrogen peroxide, showing 2.49 U/mg of specific activity in distilled water and 2.11 U/mg in water adjusted at pH 6. At this latter condition this catalyst showed 6-fold higher activity compared with Cu-CALB-BIC, 4-fold more than Cu-CALB-BIC2 and 2-fold than Cu-CALB-PHOS (Table 2). In simply distilled water, Cu-CALB-PHOS2 showed almost six times more activity than Cu-CALB-PHOS, 2 times than Cu-CALB-BIC and 4 times than Cu-CALB-BIC2.

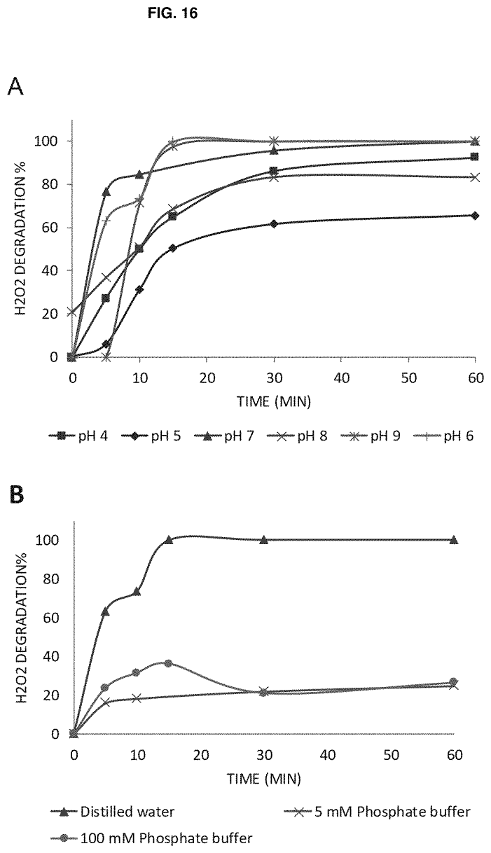

In particular, for Cu-CALB-PHOS2, the evaluation of the catalase activity was measured in distilled water at a range of pH from 4 to 9 (FIG. 16, Table 2). The best result was achieved at pH 6, and good values were obtained also at pH 4 or pH 9 (FIG. 16A) although the catalyst was instable at these conditions. Also FIG. 13B showed the negative influence of the presence of buffer in the solution, clearly decreasing the catalase activity of this cu biohybrid at these conditions.

In the case of the others Cu bionanohybrids, for example Cu-CALB-PHOS showed the highest activity value in distilled water adjusted at pH 6 whereas the best catalytic activity at higher pHs was achieved in the presence of 5 mM buffer (Table 2).

For Cu-CALB-BIC, the best results were obtained in distilled water, also adjusted at pH 6 and 7 whereas for Cu-CALB-BIC2 were in distilled water at pH 7 and 8.

In comparison with the natural catalase, the very interesting results was found in the conditions of distilled water (without pH adjust) where the Cu-CALB-PHOS2 showed similar activity than the natural enzyme.

### Conclusions of the Examples

Novel Cu nanoparticles biohybrids have been synthesized, where the control of the Cu species, size, crystallinity and morphology of the nanoparticles was possible depending on the methodology used, for example being possible to obtain bionanohybrids containing very small size Cu (0) NPs, exclusively Cu₂O NPs or crystalline Cu₃(PO₄)₂ NPs.

Cu bionanohybrids showed excellent catalytic activity in reduction of p-nitrophenol. These novel catalysts showed excellent catalytic performance in the degradation of a toxic and pollutant compound as BPA, where the best one, the Cu-CALB-PHOS2 biohybrid, was able to eliminate more than 95% of BPA (46 ppm) in 20 min in the presence of hydrogen peroxide at pH 8 using 1.5 g/L of catalyst.

Furthermore, here it has been demonstrated for the first time that these novel Cu bionanohybrids showed catalase activity, even in one case similar values of specific activity to the natural Catalase (Catazyme from Novozymes) in distilled water, which demonstrate that they are stable artificial metalloenzymes with possible interesting applications. This also can be extended to comparison with other catalases from different sources or even pseudocatalases.

1. A hybrid material comprising:
a protein matrix comprising lipase B from Candida Antarctica and
nanoparticles of copper species selected from: Cu (0), Cu₂O Cu₃(PO₄)₂ or any combination thereof,
wherein the nanoparticles have an average diameter between 3 and 15 nm and are homogeneously distributed within the matrix.

2. The hybrid material according to claim 1, wherein the protein matrix consists of lipase B from Candida antarctica.

3. The hybrid material according to claim 1, wherein the hybrid material has between 22 and 94% by weight in Cu.

4. A process for preparing the hybrid material described in claim 1, the process comprises the next steps:
a) addition under stirring of the lipase B from Candida antarctica to a buffer solution, wherein the pH of the buffer solution ranges between 6 and 10
b) addition of a copper salt to the solution obtained in step a) at a temperature between 20 and 25° C.,
c) incubation of the solution obtained in step b) for a time between 16 h and 3 days,
d) collecting, washing and drying the hybrid material obtained in the previous step.

5. The process according to claim 4, wherein the collected hybrid material is washed with water and/or drying by lyophilization in step d).

6. The process according to claim 4, wherein the process includes an additional step c)′, after step c) and before step d), which is:
a reduction step comprising the addition of a reducing agent to the mixture obtained in step c) or
an oxidation step comprising the addition of an oxidant agent, to the mixture obtained in step c).

7. The process according to claim 4, wherein the buffer solution of step a) is either a sodium phosphate buffer or a sodium bicarbonate buffer.

8. The process according to claim 4, wherein between 0.3 to 3 mg of lipase B from Candida antarctica is added per ml of buffer solution in step a).

9. The process according to claim 4, wherein the copper salt is Cu₂SO₄.5H₂O.

10. The process according to claim 4, wherein 10 mg of copper salt is added in step b) per ml of buffer solution.

11. The process according to claim 4, wherein the incubation time in step c) ranges from 16 h to 24 h.

12. The process according to claim 4, wherein the process comprises the following steps:
a) addition under stirring of the lipase B from Candida antarctica to a buffer solution of sodium bicarbonate 0.1 M, wherein the protein is added in a proportion of 0.3 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for 16 h,
c′) addition of sodium borohydride to a final concentration thereof of 0.12 M,
d) collecting, washing with water and drying by lyophilization the hybrid material obtained on the previous step.

13. The process according to claim 4 wherein the process comprises the following steps:
a) addition under stirring of the lipase B from Candida antarctica to a buffer solution of sodium phosphate 0.1 M, wherein the protein is added in a proportion of 0.3 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for 16 h,
c′) addition of sodium borohydride to a final concentration thereof of 0.12 M,
d) collecting, washing with water and drying by lyophilization the hybrid material obtained on the previous step.

14. The process according to claim 4 wherein the process comprises the following steps:
a) addition under stirring of the lipase B from Candida antarctica to a buffer solution of sodium bicarbonate 0.1 M, wherein the protein is added in a proportion of 0.6 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for 16 h,
c′) addition of sodium borohydride to a final concentration thereof of 0.12 M,
d) collecting, washing with water and drying by lyophilization the hybrid material obtained on the previous step.

15. The process according to claim 4 wherein the process comprises the following steps:
a) addition under stirring of the lipase B from Candida antarctica to a buffer solution of sodium phosphate 0.1 M, wherein the protein is added in a proportion of 0.6 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for 16 h,
c′) addition of sodium borohydride to a final concentration thereof of 0.12 M,
d) collecting, washing with water and drying by lyophilization the hybrid material obtained on the previous step.

16. The process according to claim 4 wherein the process comprises the following steps:
a) addition under stirring of the lipase B from Candida antarctica to a buffer solution of sodium phosphate 0.1 M, wherein the protein is added in a proportion of 0.3 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for a 16 h.
d) collecting, washing with water and drying by lyophilization the hybrid material obtained on the previous step.

17. The process according to claim 4 wherein the process comprises the following steps:
a) addition under stirring of the lipase B from Candida antarctica to a buffer solution of sodium phosphate 0.1 M, wherein the protein is added in a proportion of 0.3 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for a 16 h,
c′) addition of H₂O₂ to a final concentration thereof of 0.1 M,
d) collecting, washing with water and drying by lyophilization the hybrid material obtained on the previous step.

18. The process according to claim 4 wherein the process comprises the following steps:
a) addition under stirring of the lipase B from Candida antarctica, to a buffer solution of sodium phosphate 0.1 M, wherein the protein is added in a proportion of 0.3 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for a 16 h,
c′) addition of NaOH to a final concentration thereof of 0.5 M,
d) collecting, washing with water and drying by lyophilization the hybrid material obtained on the previous step.

19. The process according to claim 4 wherein the process comprises the following steps:
a) addition under stirring of the lipase B from Candida antarctica, to a buffer solution of sodium phosphate 0.1 M, wherein the protein is added in a proportion of 0.3 mg per ml of buffer solution,
b) addition of the Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for a 16 h,
c′) addition of sodium borohydride to a final concentration thereof of 0.012 M,
d) collecting, washing with water and drying by lyophilization the hybrid material obtained on the previous step.

20. The process according to claim 4 wherein the process comprises the following steps:
a) addition under stirring of the lipase B from Candida antarctica to a buffer solution of sodium phosphate 0.1 M (pH 7), wherein the protein is added in a proportion of 3 mg per ml of buffer solution,
b) addition of Cu₂SO₄.5H₂O to the solution obtained in step at 20-25° C., wherein the Cu₂SO₄.5H₂O is added in a proportion of 10 mg per ml of buffer solution,
c) incubation of the solution obtained in step b) for 3 days,
c′) optionally addition of sodium borohydride to a final concentration thereof of 0.12 M,
d) collecting, washing with water and drying by lyophilization the hybrid material obtained on the previous step.

21. A catalyst characterized in that it comprises the hybrid material described in claim 1.

22-24. (canceled)
